(19) United States
(12) Patent Application Publication
(10) Pub. No.: US 2018/0146698 A1
(43) Pub. Date: May 31, 2018

(54) FORMULA FEED FOR POULTRY

(71) Applicant: JXTG Nippon Oil & Energy Corporation, Chiyoda-ku, Tokyo (JP)

(72) Inventors: Wataru Sato, Tokyo (JP); Hidetada Nagai, Tokyo (JP); Yuki Kawashima, Tokyo (JP); Michihisa Ikarashi, Tokyo (JP); Yutaka Sakai, Tokyo (JP)

(21) Appl. No.: 15/575,965

(22) PCT Filed: May 27, 2016
(86) PCT No.: PCT/JP2016/065661

§ 371 Date: Nov. 21, 2017

(30) Foreign Application Priority Data
May 29, 2015 (JP) 2015-110258

(51) Int. Cl. A23K 20/179; A23K 10/30; A23K 50/75; A23K 10/16; A61K 31/122

(52) U.S. Cl. CPC A23K 20/179; A23K 10/30; A61K 31/122; A23K 10/16; A23K 50/75

(57) ABSTRACT

A method of obtaining eggs taking on the yolk color preferred by consumers, whereby cost reduction and stable supply of feeds can be achieved, is provided. Specifically, the present invention provides a formula feed for poultry, in which the content of corn is 50% or less and the content of astaxanthin from a dry powder of a bacterium is 1 to 8 ppm, and a method of obtaining eggs taking on a desired yolk color using the formula feed. According to the present invention, it becomes possible to provide a formula feed that can be stably supplied at low price without influence of seasonal and weather conditions. In addition, poultry eggs taking on a yolk color that satisfies the consumer preference can be provided at a lower price.

### TECHNICAL FIELD

The present invention relates to a formula feed for poultry. More specifically, it relates to an astaxanthin-containing formula feed for poultry.

### BACKGROUND ART

Eggs of poultry such as chickens are nutritious and widely used directly as a food or material for a variety of confectionery and food products.

Formula feeds with various compositions are known as poultry feeds. In most cases, feeds mainly containing corn are used. One important factor that determines marketability of eggs is the yolk color. It is well-known that the yolk color is tinged with yellow because colorants such as carotenoids contained in feeds, and in particular, colorants such as lutein and zeaxanthin contained in corn, are absorbed by laying chickens and transferred to and accumulated in eggs.

Meanwhile, in recent years, consumers tend to prefer eggs with darker yolk colors or eggs that are more tinged with red. In general, formula feeds supplied to laying hens contain corn at a proportion of about 50% to 60%. However, colors that satisfy the consumer preference cannot be achieved using only cereals such as corn. Therefore, color enhancers containing carotenoids as the major components are added to most of feeds at present. Examples of color enhancers used include paprika-derived colorants and astaxanthin (e.g., Patent Literature 1 to 3).

### CITATION LIST

### Patent Literature

Patent Literature 1: JP Patent Publication (Kokai) No. H7-143864 A (1995)

Patent Literature 2: JP Patent Publication (Kokai) No. H7-115915 A (1995)

Patent Literature 3: JP Patent Publication (Kokai) No. H8-242774 A (1996)

### SUMMARY OF INVENTION

### Technical Problem

There is a high demand on corn because it is used not only for poultry but also for a variety of feeds. In addition, the supply of corn may become insufficient because of the influence of poor harvest caused by abnormal weather such as drought. For such reasons, the price of corn may significantly vary, which might result in a price increase. In order to cope with such case, the use of feeds with low corn contents has been under consideration. However, it has been considered that corn is an essential component of formula feeds for laying chickens in order to provide eggs taking on the yolk color preferred by consumers.

In addition, it is known that the situation of supplying paprika varies by seasonal and weather conditions since paprika used for color enhancers is a plant, and therefore, the price of paprika would vary by season.

Hence, there is a demand to reduce the cost, decrease the price range, and use feeds that can be stably supplied as well as to provide eggs taking on the yolk color preferred by consumers.

### Solution to Problem

The group of the present inventors has established the technology of producing astaxanthin with the use of bacteria. The use of this technology allows stable supply of astaxanthin through a whole year. It has been reported that astaxanthin has antioxidation effects, which are about 500-fold stronger in terms of singlet oxygen quenching activity and about 1000-fold stronger in terms of lipid peroxidation inhibition activity, compared with vitamin E. It is therefore possible to mix astaxanthin in a feed with an expectation that astaxanthin will exert antioxidation effects as well as feed color enhancer effects.

In consideration of the aforementioned problems, the present inventors made various researches on the probability of using, as a formula feed for poultry, a feed with a corn concentration lower than a standard corn concentration of 50% to 60%. As a result, surprisingly, the present inventors found that it is possible to achieve a desired color tone with the use of astaxanthin from a dry powder of a bacterium as a color enhancer when adding the color enhancer to a low-corn-content feed in an amount lower than the addition amount in a conventional high-corn-content feed, thereby enabling to reduce the addition amount of the color enhancer.

It was confirmed that this effect is unique to astaxanthin but not to trans-capsanthin (t-cap) that is a paprika-derived colorant. It was therefore revealed that it is possible to reduce the cost of a formula feed and achieve a desired yolk color by reducing the corn content in a feed and the addition amount of astaxanthin serving as a color enhancer.

Specifically, the present invention encompasses the following [1] to [9].

[1] A formula feed for poultry, in which the content of corn is 50% or less and the content of astaxanthin from a dry powder of a bacterium is 1 to 8 ppm.
[2] The formula feed according to [1], wherein the bacterium is a microorganism of the genus Paracoccus.
[3] The formula feed according to [1] or [2], wherein the content of rice, wheat, barley, soybean, milo, and/or raw materials therefrom is 10% or more.
[4] A method of obtaining eggs taking on a yolk color corresponding to a color fan value of 9 to 15 by raising poultry using a formula feed, in which the content of corn is 50% or less and the content of astaxanthin from a dry powder of a bacterium is 1 to 8 ppm.
[5] The method according to [4], which comprises supplying the formula feed for 2 weeks or longer.
[6] The method according to [4] or [5], wherein the bacterium is a microorganism of the genus Paracoccus.
[7] A poultry egg taking on a yolk color corresponding to a color fan value of 9 to 15, which is obtained by raising poultry using a formula feed, in which the content of corn is 50% or less and the content of astaxanthin from a dry powder of a bacterium is 1 to 8 ppm.
[8] The poultry egg according to [7], wherein the bacterium is a microorganism of the genus Paracoccus.
[9] The poultry egg according to [7] or [8], wherein the concentration of astaxanthin in the yolk is 12 ppm or less.

This description includes part or all of the content as disclosed in the description and/or drawings of Japanese Patent Application No. 2015-110258, which is a priority document of the present application.

### Advantageous Effects of Invention

According to the present invention, it becomes possible to reduce the amount of corn mixed in a formula feed for poultry and the addition amount of a color enhancer, thereby reducing the cost and at the same time providing a formula feed that can be stably supplied without influence of seasonal and weather conditions. As a result, poultry eggs taking on a yolk color that satisfies the consumer preference can be provided at low price.

### BRIEF DESCRIPTION OF THE DRAWINGS

FIG. 1 shows the relationship between the measured values of astaxanthin concentration in each feed and the yolk color fan value (CF value) in the case of adding astaxanthin to the feed at a concentration of 1 to 8 ppm: ♦: standard-corn-content feed; ▪: low-corn-content feed.

FIG. 2 shows the relationship between the measured values of t-capsanthin concentration in each feed and the yolk color fan value (CF value) in the case of adding t-capsanthin to the feed at a concentration of 1 to 8 ppm: ♦: standard-corn-content feed; ▪: low-corn-content feed.

FIG. 3 shows the yolk carotenoid concentration and the carotenoid composition in eggs collected from chickens fed with either a feed in which the astaxanthin concentration is 2 ppm or 4 ppm or a feed in which the t-capsanthin concentration is 2 ppm or 4 ppm.

FIG. 4 shows the results of the yolk astaxanthin concentration in the case of adding astaxanthin to a feed at a concentration of 1 to 8 ppm, compared between a standard-corn-content feed and a low-corn-content feed: □: standard-corn-content feed; ▪: low-corn-content feed.

FIG. 5 shows the results of the yolk t-capsanthin concentration in the case of adding t-capsanthin to a feed at a concentration of 1 to 8 ppm, compared between a standard-corn-content feed and a low-corn-content feed: □: standard-corn-content feed; ▪: low-corn-content feed.

FIG. 6 shows the relationship between the yolk astaxanthin concentration and the CF value in the case of adding astaxanthin to a feed: ♦: standard-corn-content feed; ▪: low-corn-content feed.

FIG. 7 shows the relationship between the yolk t-capsanthin concentration and the CF value in the case of adding t-capsanthin to a feed: ♦: standard-corn-content feed; ▪: low-corn-content feed.

FIG. 8a shows the relationship between the measured values of astaxanthin or t-capsanthin concentration in a feed with a corn content of 30% and the yolk color fan value in the case of adding astaxanthin or t-capsanthin to the feed at a concentration of 1 to 16 ppm. Curve fitting was carried out by the least-square method using the following approximation formula: CF=b×(1−EXP(−1×a×colorant concentration in the feed))+intercept (♦: astaxanthin; ▪: t-capsanthin).

FIG. 8b shows the relationship between the measured values of astaxanthin or t-capsanthin concentration in a feed with a corn content of 50% and the yolk color fan value in the case of adding astaxanthin or t-capsanthin to the feed at a concentration of 1 to 16 ppm. Curve fitting was carried out by the least-square method using the following approximation formula: CF=b×(1−EXP(−1×a×colorant concentration in the feed))+intercept (♦: astaxanthin; ▪: t-capsanthin).

FIG. 9a shows the L* values determined by using a colorimeter for measuring the yolk color of eggs obtained in the case of adding astaxanthin or t-capsanthin to a feed with a corn content of 30% to 50% at a concentration of 1 to 16 ppm.

FIG. 9b shows the a* values determined by using a colorimeter for measuring the yolk color of eggs obtained in the case of adding astaxanthin or t-capsanthin to a feed with a corn content of 30% to 50% at a concentration of 1 to 16 ppm.

FIG. 9c shows the b* values determined by using a colorimeter for measuring the yolk color of eggs obtained in the case of adding astaxanthin or t-capsanthin to a feed with a corn content of 30% to 50% at a concentration of 1 to 16 ppm.

FIG. 10 shows the yolk carotenoid concentration and the carotenoid composition in eggs collected from chickens fed with a formula feed with a corn content of 30% to 50% in which astaxanthin or t-capsanthin is added at a concentration of 1 to 16 ppm.

FIG. 11 shows the yolk color fan values for eggs collected from chickens fed with a formula feed with a corn content of 0% to 30% in which astaxanthin or t-capsanthin is added at a concentration of 2 ppm or 4 ppm.

FIG. 12a shows the relationship between the corn content (%) and the yolk color fan value in the case of adding astaxanthin or t-capsanthin to a feed with a corn content of 0% to 30% at a concentration of 2 ppm: ♦: astaxanthin; ▪: t-capsanthin.

FIG. 12b shows the relationship between the corn content (%) and the yolk color fan value in the case of adding astaxanthin or t-capsanthin to a feed with a corn content of 30% to 50% at a concentration of 2 ppm: ♦: astaxanthin; ▪: t-capsanthin.

FIG. 12c shows the relationship between the corn content (%) and the yolk color fan value in the case of adding astaxanthin or t-capsanthin to a feed with a corn content of 0% to 30% at a concentration of 4 ppm: ♦: astaxanthin; ▪: t-capsanthin.

FIG. 13 shows the yolk carotenoid concentration and the carotenoid composition in eggs collected from chickens fed with a formula feed with a corn content of 0% to 30% in which astaxanthin or t-capsanthin is added at a concentration of 2 ppm or 4 ppm.

FIG. 14a shows the L* values determined by using a colorimeter for measuring the yolk color of eggs obtained in the case of adding astaxanthin or t-capsanthin to a feed with a corn content of 0% to 30% at a concentration of 2 ppm or 4 ppm.

FIG. 14b shows the a* values determined by using a colorimeter for measuring the yolk color of eggs obtained in the case of adding astaxanthin or t-capsanthin to a feed with a corn content of 0% to 30% at a concentration of 2 ppm or 4 ppm.

FIG. 14c shows the b* values determined by using a colorimeter for measuring the yolk color of eggs obtained in the case of adding astaxanthin or t-capsanthin to a feed with a corn content of 0% to 30% at a concentration of 2 ppm or 4 ppm.

### DESCRIPTION OF EMBODIMENTS

At present, the Zen-Noh Yolk Color Chart (CF) by JA Z-Tamago Co., Ltd. and the Roche Yolk Color Fan (RYCF) by Roche are used for yolk color measurement of chicken eggs or the like in an ordinary method, which is the most common yolk color evaluation method in the art. The color fan value (CF value) ranges from 1 to 15. At present, color fan value measurement is carried out by automatic measurement using a device capable of electronically determining the color tone (e.g., Egg Multitester EMT-7300 (JA Z-Tamago Co., Ltd.)).

In addition, the yolk color is measured using a colorimeter in some cases. For example, the a* value of the yolk with a CF value of 10 is about 8, the a* value of the yolk with a CF value of 12 is about 12, and the a* value of the yolk with a CF value of 14 is about 15. Such values, however, may vary depending on feed components, chicken varieties, colorimeter measurement methods, and the like. For such reasons, there is no clear coordination between the colorimeter measurement value and the CF value at this time. Therefore, the CF value is an objective measurement value that can be most commonly understood among those skilled in the art.

The term “poultry” used herein refers to chickens, quails, turkeys, guineafowls, pigeons, ducks, geese, or the like. The term “poultry eggs” used herein refers to eggs obtained therefrom. In many countries and especially in Japan, the most consumed eggs are chicken eggs. Therefore, the present invention has been made using manly chickens and chicken eggs, and the present invention can be carried out preferably using chickens. However, the present invention is not limited to chickens and chicken eggs. The terms such as “laying chickens” and “eggs” can be applied to the above-mentioned “poultry” in general.

The values with the units “%” and “ppm” which represent the content and the addition amount, respectively, used herein are each intended to mean a proportion by weight.

When a formula feed with a corn content of 50% to 60% is used without a color enhancer, the color fan values of the obtained eggs are about 6 to 9. Individual preference of yolk color differs among people as well as countries. In recent years, eggs taking on a yolk color with an increased color fan value of, for example, 12 to 14 or even 15 are considered to be preferred in some case. Accordingly, in order to provide eggs that satisfy the consumer preference, it is essential to add a colorant so as to obtain eggs taking on a color darker (i.e., eggs with a high color fan value) than the yolk color that can be realized with a corn colorant alone.

For instance, in a case where the corn content in a formula feed is reduced to 10% or less, the color fan value of the obtained eggs falls within a range of about 1 to 6 (6 is a value extrapolated from an approximate curve) because of reduction in the amounts of yellow colorants lutein and zeaxanthin.

The present invention provides a formula feed for poultry, characterized in that astaxanthin from a dry powder of a bacterium is added to a feed with a corn content of 50% or less so as to result in an astaxanthin concentration of 1 to 8 ppm. The corn content of the formula feed of the present invention may be 50% or less, 40% or less, 30% or less, 20% or less, 10% or less, 8% or less, 5% or less, or 3% or less. It is also possible not to mix corn in the formula feed (i.e., a corn content of 0%). However, the corn content may be, for example, 1% or more, 2% or more, 3% or more, 10% or more, 20% or more, or 30% or more, depending on a desired color fan value or the like, and may be adjusted appropriately. The color enhancement improving effects of astaxanthin can be confirmed in a feed with a corn content of 50% or less, and the color enhancement improving effects of the present invention are observed depending on a decrease in the corn content.

Bacteria that can be used in the present invention are not limited as long as they are bacteria capable of producing astaxanthin. Examples of the bacteria that can be used include bacteria of the genus Paracoccus, bacteria of the genus Sphingomonas, bacteria of the genus Brevundimonas, and bacteria of the genus Erythrobacter. Preferably, bacteria of the genus Paracoccus are used. Examples of bacteria of the genus Paracoccus include Paracoccus carotinifaciens, Paracoccus marcusii, Paracoccus haeundaensis, Paracoccus zeaxanthinifaciens, Paracoccus denitrificans, Paracoccus aminovorans, Paracoccus aminophilus, Paracoccus kourii, Paracoccus halodenitrificans, and Paracoccus alcaliphilus. Particularly preferably, Paracoccus carotinifaciens can be used. Examples of strains of Paracoccus carotinifaciens include the Paracoccus carotinifaciens E-396 strain (FERM BP-4283).

A mutant strain having a modified ability to produce astaxanthin may be used in the present invention. Examples of such mutant strain include, but are not limited to, a strain that is highly capable of producing astaxanthin (JP Patent Publication (Kokai) No. 2001-95500 A).

A method for culturing an astaxanthin-producing bacterium is not particularly limited. For instance, the following method using, as a medium, a medium containing, for example, a carbon source, a nitrogen source, an inorganic salt, and optionally, a special necessary nutrient (e.g., a vitamin, amino acid, or nucleic acid), which are required for the growth of the bacterium, is employed.

Examples of a carbon source include: sugars such as glucose, sucrose, fructose, trehalose, mannose, mannitol, and maltose; organic acids such as acetic acid, fumaric acid, citric acid, propionic acid, malic acid, and malonic acid; alcohols such as ethanol, propanol, butanol, pentanol, hexanol, and isobutanol; and combinations thereof. The proportion of a carbon source to be added depends on the type thereof; however, it can be usually 1 to 100 g (e.g., 2 to 50 g) in 1 L of medium.

Examples of a nitrogen source include potassium nitrate, ammonium nitrate, ammonium chloride, ammonium sulfate, ammonium phosphate, ammonia, urea, and combinations thereof. The proportion of a nitrogen source to be added depends on the type thereof; however, it can be usually 0.1 to 20 g (e.g., 1 to 10 g) in 1 L of medium.

Examples of an inorganic salt include potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, zinc chloride, copper sulfate, calcium chloride, calcium carbonate, sodium carbonate, and combinations thereof. The proportion of an inorganic salt to be added depends on the type thereof; however, it can be usually 0.1 mg to 10 g in 1 L of medium.

Examples of a special necessary nutrient include vitamins, nucleic acids, yeast extract, peptone, meat extract, malt extract, corn steep liquor, dried yeast, soybean cake, soybean oil, olive oil, corn oil, linseed oil, and combinations thereof. The proportion of a special necessary nutrient to be added depends on the type thereof; however, it can be generally 0.01 mg to 100 g in 1 L of medium.

The medium pH is adjusted to, for example, pH 2 to 12 or pH 6 to 9.

It is possible to perform culture by shake culture or aeration culture at, for example, 10° C. to 70° C. (e.g., 20° C. to 35° C.) for usually 1 to 20 days (e.g., 2 to 9 days). The astaxanthin-producing bacterium is cultured under such conditions. As a result of culture, bacterial cells of the bacterium intracellularly or extracellularly produce a large amount of astaxanthin.

A culture solution obtained by the above culture method can be appropriately concentrated. Examples of a concentration method include membrane concentration and centrifugation.

Following the aforementioned step, medium components are removed. Upon centrifugation, if concentration takes place, water is added to the resulting concentrated liquid so as to remove medium components. If membrane separation is employed, diafiltration is performed so as to remove medium components. The amount of water added may be approximately 1 to 5 times that of the concentrated liquid, although the amount would vary depending on the colorant content or the like in the concentrated liquid.

Further, a culture solution or a concentrate is dried in order to obtain a dry powder. In the present invention, it is possible to use a powder formed by drying astaxanthin-containing bacterial cells, which are obtained in the form of a culture solution or bacterial cell slurry.

A drying method is not particularly limited. Known drying methods such as spray drying, spray granulation drying, drum drying, freeze-drying, and fluidized-bed drying can be used. In this manner, an astaxanthin-containing dry powder can be produced. In addition, a powder prepared by further reducing the particle size of the obtained dry powder by pulverization, for example, a powder having a particle size of 1 μm to 30 μm, 1 μm to 20 μm, 5 μm to 20 μm, or 7 μm to 20 μm can be used.

In one embodiment, a dry powder suitably used for the present invention is formed with dried bacterial cells obtained by a production method comprising a step of bringing bacterial cells capable of producing astaxanthin into contact with a heat transfer unit at more than 100° C. for drying via heat transfer. For instance, when the dry powder has a volume particle size (D50) of 7 to 12 μm, the change of the diffusion coefficient D of astaxanthin extracted via ethanol extraction can be represented by the quotient (i.e., b₂₅/b₃₅) 0.807±0.05, as the result of dividing the diffusion coefficient D determined at 25° C. by the diffusion coefficient D determined at 35° C.

It is possible to purchase commercially available PANAFERD-P (JX Energy Group) as astaxanthin that can be used in the present invention.

The above dry powder is added to a feed with a corn content of 50% or less in order to prepare the formula feed for poultry of the present invention. The term “corn” in the expression “corn content” used herein mainly refers to dried and pulverized corn grains and corn-derived processed products such as corn gluten feed, corn gluten meal, and dried distiller's grains with solubles (DDGS) (i.e., corn distillation cake).

With the use of the formula feed for poultry of the present invention, it becomes possible to obtain eggs having a desired color fan value, in which the yolk carotenoid concentration is even lower than the yolk carotenoid concentration in eggs obtained from poultry fed with a high-corn-content feed supplemented with the above astaxanthin-containing dry powder.

According to the method of the present invention, astaxanthin-containing poultry eggs are obtained by feeding poultry with a formula feed prepared by adding the above dry powder to a feed with a corn content of 50% or less, raising the poultry, and collecting eggs.

Although the content of astaxanthin in the astaxanthin-containing dry power of a bacterium may vary depending on the type of bacterium, culture method, and the like, it may be 1 to 30 mg per 1 g of the powder. For instance, 1 g of a dry powder from one strain of the genus Paracoccus may contain about 2.1 mg to 2.5 mg (2,100 to 2,500 ppm) of astaxanthin. 1 g of a dry powder from a different strain of the genus Paracoccus may contain about 20 mg to 25 mg (20,000 to 25,000 ppm) of astaxanthin. According to the present invention, a formula feed obtained as a final product may contain astaxanthin at a concentration of 1 to 8 ppm (0.1 to 0.8 mg per 100 g of the feed), for example, 1 ppm, 2 ppm, 3 ppm, 4 ppm, 5 ppm, 6 ppm, 7 ppm, 8 ppm, 1 to 4 ppm, or 2 to 4 ppm. Therefore, the astaxanthin-containing dry powder of a bacterium obtained as described above may be added to 100 g of a feed so that the astaxanthin concentration falls within a range of about 3 to 800 mg (30 to 8,000 ppm), for example, 4 to 400 mg (40 to 4,000 ppm) or 4 to 40 mg (40 to 400 ppm).

It is also possible to preliminarily mix the astaxanthin-containing dry powder of a bacterium with vitamins and the like in a premixed product.

The period of feeding with a formula feed, to which the astaxanthin-containing dry powder of a bacterium has been added, may be 2 weeks or more, 3 weeks or more, or 4 weeks or more before egg laying.

The astaxanthin concentration in the yolk of eggs obtained by feeding with the formula feed for poultry containing the astaxanthin-containing dry powder of a bacterium according to the present invention may be, for example, 12 ppm or less, 10 ppm or less, 9 ppm or less, 8 ppm or less, or 7 ppm or less. It may also be 0.1 ppm or more, 0.3 ppm or more, 0.5 ppm or more, 0.8 ppm or more, or 1 ppm or more.

According to the method of the present invention, eggs taking on a yolk color corresponding to a color fan value of 9 to 15 can be obtained when the astaxanthin concentration in the feed is 1 ppm to 8 ppm. It is also possible to obtain eggs taking on a yolk color corresponding to a color fan value of up to 14 by adding astaxanthin at a concentration of 4 ppm or less. As the desired color fan value differs depending on the consumer preference, intended use of eggs, or the like, the ideal content (concentration) of astaxanthin to be added to the formula feed can be adjusted for obtaining eggs having an intended color fan value, for example, a color fan value of 9, 10, 11, 12, 13, 14, or 15.

In the poultry fed with the formula feed of the present invention, when a low-corn-content feed is used, the content of carotenoid transferred to the yolk is low and the astaxanthin concentration is 10 ppm or less (1 mg or less per 100 g). Nevertheless, the poultry produce eggs taking on a yolk color corresponding to a color fan value of 9 to 15. In general, when a high-corn-content formula feed is used, the carotenoid content in the obtained yolk is about 10 to 30 ppm. Therefore, it is surprising that the desired color fan value can be achieved using the composition of the formula feed of the present invention.

While without wishing to be bound by any theory, the reason for unexpected effects of the present invention is considered to be that zeaxanthin and lutein, which are yellow carotenoids in corn, prevent astaxanthin from being absorbed and accumulated in the yolk in a competitive manner, indicating that reduction of the amounts of such colorants causes astaxanthin to be absorbed and accumulated in the yolk to a greater extent. The chemical structure of astaxanthin is very similar to the chemical structures of zeaxanthin and lutein. The effects that are considered to be due to reduction in the amounts of zeaxanthin and lutein absorbed or accumulated are not observed for t-capsanthin that is a paprika-derived colorant.

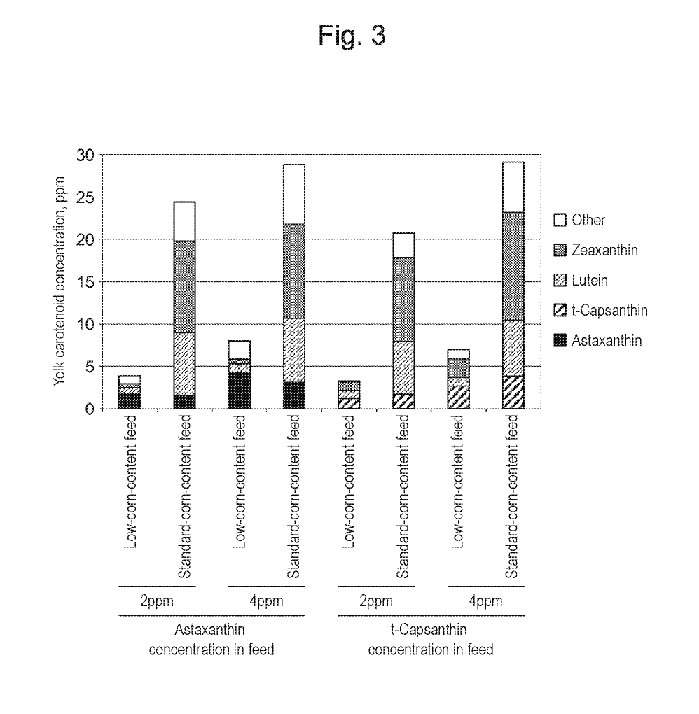

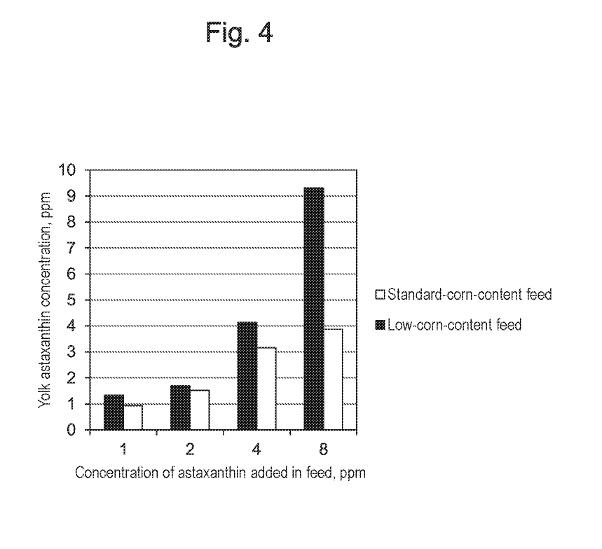

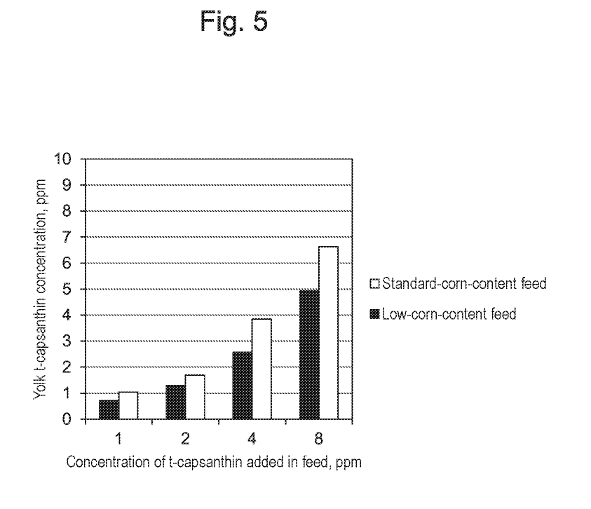

Astaxanthin was added in the same amount to a high-corn-content feed (corn content: more than 50%) and a low-corn-content feed (corn content: 10% or less) to compare the yolk carotenoid composition. As a result, in the case of the low-corn-content feed, it was revealed that, although the total carotenoid amount is low, the astaxanthin concentration was high and the color fan value was also high (FIGS. 3 and 4 and Table 4). Meanwhile, when t-capsanthin was added, the t-capsanthin concentration was higher in the case of giving the high-corn-content feed (FIG. 5). In addition, although the concentration of t-capsanthin among carotenoids relatively increased, the color fan value did not increase (Table 4).

The dry powder of an astaxanthin-producing bacterium contains several other carotenoids, in addition to astaxanthin. For instance, the content of carotenoids in a dry powder of a bacterium of one Paracoccus strain is about 3%, and astaxanthin accounts for about 60% thereof, that is to say, about 2% with respect to the total amount of the dry powder of the bacterium. Therefore, the above results are considered to suggest a probability that combined effects of all carotenoids including astaxanthin (also including metabolites and precursors of astaxanthin) and additional components contained in the dry powder of the bacterium can be obtained.

When reducing the content of corn in the feed, it is necessary to supplement nutrients other than a colorant obtained from corn with the use of other materials. Examples of materials that can replace corn include carbohydrates such as rice, wheat, barley, soybean, milo, and/or raw materials derived therefrom, which have low yellow carotenoid contents. The contents thereof may be, for example, 10% or more, 20% or more, 30% or more, 40% or more, or 50% or more. For example, brown rice, white rice, rice bran, or the like can be used as rice. In addition, the amounts of amino acids, vitamins, minerals, and the like to be added can be adequately adjusted, if necessary. Those skilled in the art can prepare a formula feed with an appropriate composition without affecting egg production and the like, even when its corn content is lowered.

### EXAMPLES

The present invention is described in further details with reference to the Examples below. However, the present invention is not limited thereto.

### [Example 1] Chicken Egg Color Enhancement Test 1

A preparation of dry cells of a Paracoccus bacterium (with an astaxanthin content of 2%) and a paprika colorant preparation (with a t-capsanthin content of 0.25%) were used as coloring agents for evaluation.

Cells of a nitrosoguanidine mutant strain obtained from the Paracoccus carotinifaciens E-396 strain (FERM BP-4283) were used as carotenoid-producing Paracoccus bacterial cells for a preparation of dry cells of a Paracoccus bacterium. The preparation was cultured in a seed flask medium and then cultured in a main culture medium at 28° C. under aerobic conditions until the bacterial cell concentration reached the maximum level. Next, the cultured cells were collected and recovered using a centrifuge.

The recovered Paracoccus bacterial cells were dried using a double drum dryer at a drum rotation speed of 3.5 rpm and a drum temperature of 140° C. The average particle size (volume particle size D50) of the obtained power was approximately 100 to 125 m. The powder was further finely powderized (pulverized) using a jet mill (Seishin Enterprise Co., Ltd.) so that the average particle size (D50) became 9 μm. The obtained product was used as a preparation of dry cells of a Paracoccus bacterium.

As a paprika colorant preparation, Color Up (a product with a total xanthophyll concentration of 5 g/kg, Kohkin Chemical Co., Ltd.) was used.

The above preparations were added to a low-corn-content feed and a standard-corn-content feed listed in Table 1, thereby preparing formula feeds each containing astaxanthin or t-capsanthin at a final concentration of 1, 2, 4, or 8 ppm. Table 2 lists measured concentrations of astaxanthin and t-capsanthin contained in the actually produced formula feeds.

TABLE 1
Basic feed composition

| | Low-corn-content | Standard-corn-content |
|---|---|---|
| Raw material | feed (%) | feed (%) |
| Corn | 8.36 | 53.29 |
| Grain sorghum | 20 | 5 |
| Soybean cake | 23 | 20.17 |
| Fish meal (CP 65%) | 2 | — |
| Fish meal (CP 60%) | — | 1.5 |
| Corn gluten meal | — | 2.85 |
| Brown rice | 30 | 3.2 |
| Defatted rice bran | 4 | 2 |
| DL-methionine | 0.04 | 0.11 |
| Vegetable oil and fat | 2.41 | — |
| Animal oil and fat | — | 2 |
| Dicalcium phosphate | 0.83 | 1.1 |
| Calcium Carbonate | 8.75 | 8.17 |
| Vitamin, Mineral | 0.61 | 0.61 |

TABLE 2
Measured concentrations of astaxanthin and t-capsanthin in feed

| | Concentration | | | |
|---|---|---|---|---|
| | astaxanthin of | | Concentration of t- | |
| | in feed, ppm | | capsanthin in feed, ppm | |
| Concentration of | Low-corn- | Standard- | Low-corn- | Standard- |
| colorant added to | content | corn-content | content | corn-content |
| feed, ppm | feed | feed | feed | feed |
| 1 | 0.9 | 1.0 | 0.6 | 1.2 |
| 2 | 1.5 | 1.9 | 1.9 | 2.0 |
| 4 | 3.3 | 3.5 | 3.5 | 4.7 |
| 8 | 7.0 | 7.1 | 7.8 | 8.5 |

Julia Light hens were used for tests. Ten chickens were provided for each test plot. The acclimatization period was set to 2 weeks. Eggs collected in Week 2, during which the color fan value became stabilized, or later, were evaluated.

Eggs were collected from each test plot. The color fan values of 10 chicken eggs were measured using Egg Multitester EMT-7300 (JA Z-Tamago Co., Ltd.) for each test plot. The yolk carotenoid concentration was determined for 3 eggs for each test plot.

Carotenoid quantities in the yolk and each feed were determined via high performance liquid chromatography (HPLC) as described below.

Two columns (Inertsil SIL-100A, 5 μm (ϕ 4.6×250 mm), GL Sciences) were connected in series for use. Elution was carried out with the introduction of an n-hexane/tetrahydrofuran/methanol liquid mixture (40:20:1) as a mobile phase at a constant temperature (around room temperature) and a flow rate of 1.0 mL per minute. Upon determination, samples were each dissolved in tetrahydrofuran and appropriately diluted with the mobile phase such that injection volume was adjusted to 20 μL. The detection for column eluate was carried out at a wavelength of 470 nm.

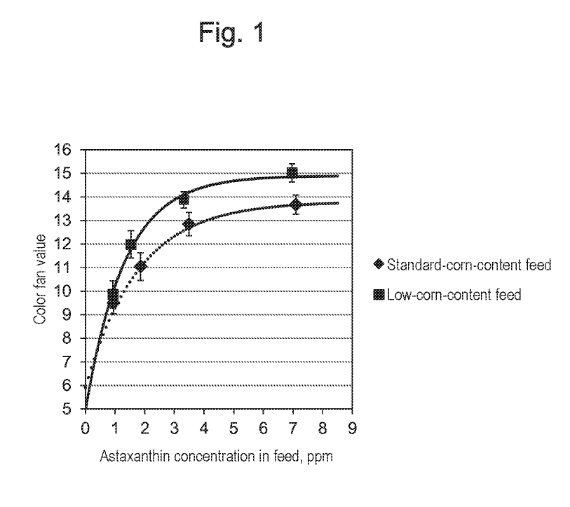

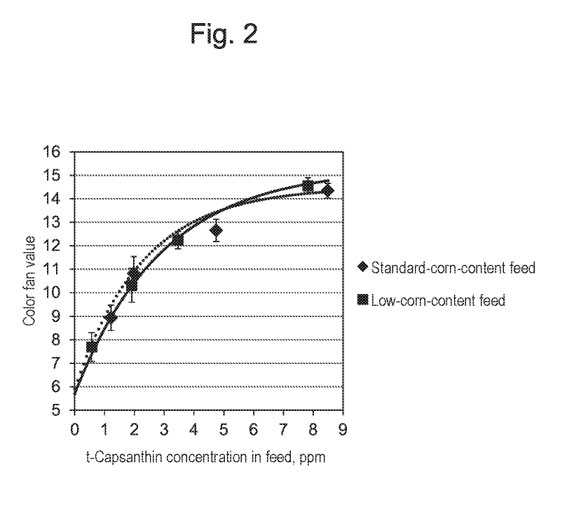

FIGS. 1 and 2 show the relationship between the concentrations of astaxanthin and t-capsanthin in both feeds and the yolk color fan values of the obtained eggs.

As a result, it was surprisingly found that, in the case of using astaxanthin as a coloring agent, the color fan value obtained using the low-corn-content feed with an astaxanthin concentration corresponding to approximately 50% to 70% of the astaxanthin concentration in the standard-corn-content feed, was comparable to that obtained using the standard-corn-content feed (FIG. 1). Meanwhile, in the case of using t-capsanthin, a decrease in the necessary amount of a coloring agent observed for astaxanthin was not observed (FIG. 2). In addition, a comparison using the standard-corn-content feed revealed that the amounts of astaxanthin and t-capsanthin required for color enhancement were comparable.

Based on the results of FIGS. 1 and 2, the concentrations of astaxanthin and t-capsanthin to be added to the low-corn-content feed in order to achieve a desired color fan value of 10 to 15 were calculated (Table 3). For example, in order to achieve a color fan value of 13, astaxanthin may be added to the low-corn-content feed so as to result in a final concentration of 2.2 ppm. Meanwhile, t-capsanthin needs to be added so as to result in a final concentration of 4.3 ppm. It was thus revealed that a desired color fan value can be achieved using astaxanthin in an amount approximately 51% relative to t-capsanthin.

TABLE 3
Comparison of colorant concentration necessary
for achieving the desired CF value

| | Colorant concentration in feed | | |
|---|---|---|---|
| Desired CF | (ppm) | | Astaxanthin/t-Capsanthin |
| value | Astaxanthin | t-Capsanthin | ratio |
| 10 | 1.0 | 1.8 | 0.56 |
| 11 | 1.3 | 2.4 | 0.54 |
| 12 | 1.7 | 3.2 | 0.53 |
| 13 | 2.2 | 4.3 | 0.51 |
| 14 | 3.2 | 6.0 | 0.53 |
| 15 | 7.0 | — | — |

The above results revealed that it is possible to reduce the amount of a coloring agent required for color enhancement by choosing a combination of astaxanthin and a low-corn-content feed. It was also confirmed in this Example that there is no significant difference in egg production, indicating that egg productivity can be maintained.

### [Example 2] Comparison of Yolk Carotenoid Content and Composition 1

The yolk carotenoid concentration of eggs obtained in Example 1 was determined by the method described in Example 1 for three eggs for each test plot. FIG. 3 shows the yolk carotenoid compositions of eggs collected from chickens that had been fed with a feed containing a colorant at a concentration of 2 ppm or 4 ppm for 4 to 6 weeks.

As is apparent from the figure, the total yolk carotenoid concentration was 20 to 30 ppm in the case of the standard-corn-content feed, while the total carotenoid concentration was 10 ppm or less in the case of the low-corn-content feed. It was confirmed that the amounts of the corn-derived colorants decreased.

In each plot for which astaxanthin was supplied, an increase in the yolk astaxanthin concentration was observed in the case of adding astaxanthin at 2 ppm or 4 ppm to the low-corn-content feed, compared with the case of adding astaxanthin to the standard-corn-content feed. Meanwhile, in the plot for which t-capsanthin was supplied, an increase in the concentration observed for astaxanthin was not observed.

The above results suggested that the amount of astaxanthin absorbed and/or accumulated in the yolk increases in relation to a decrease in the corn content in a feed.

Table 4 shows the total yolk carotenoid concentration and color fan value obtained in the case of adding 4 ppm colorant.

TABLE 4
CF value obtained when adding colorant at 4 ppm

| | Addition of astaxanthin | | Addition of t-capsanthin | |
|---|---|---|---|---|
| | Carotenoid | CF | Carotenoid | CF |
| | concentration | value | concentration | value |
| Standard-corn- | 28.9 | 12.8 | 29.2 | 12.7 |
| content feed | | | | |
| Low-corn-content | 7.9 | 13.9 | 7.0 | 12.2 |
| feed | | | | |

As is apparent from the results of Table 4, although the total yolk carotenoid concentration significantly decreased to 10 ppm or less in the feed to which astaxanthin had been added, the color fan value increased and a color fan value of approximately 14 was achieved. Meanwhile, in the case of adding t-capsanthin, the color fan value did not increase.

### [Example 3] Comparison of Colorant Concentration in the Yolk

The standard-corn-content feed and the low-corn-content feed were compared in terms of the colorant concentration in the yolk when the coloring agents used in Example 1 were added to each feed at concentrations of 1 to 8 ppm. The results were shown in FIGS. 4 and 5.

As shown in FIG. 4, in the case of adding astaxanthin, at any concentration of astaxanthin added, the yolk astaxanthin concentration in eggs obtained from chickens fed with the low-corn-content feed was greater than that in eggs obtained from chickens fed with the standard-corn-content feed, indicating that a lower corn content results in a greater amount of astaxanthin accumulated in the yolk. Meanwhile, in the case of adding t-capsanthin (FIG. 5), it was confirmed that the accumulated t-capsanthin concentration increased as the corn content increased, indicating the results opposite to those obtained for astaxanthin.

### [Example 4] Correlation Between the Colorant Concentration in the Yolk and the Color Fan Value

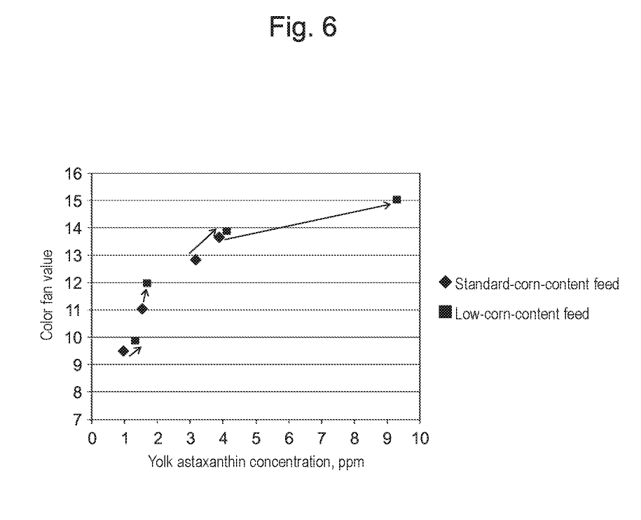

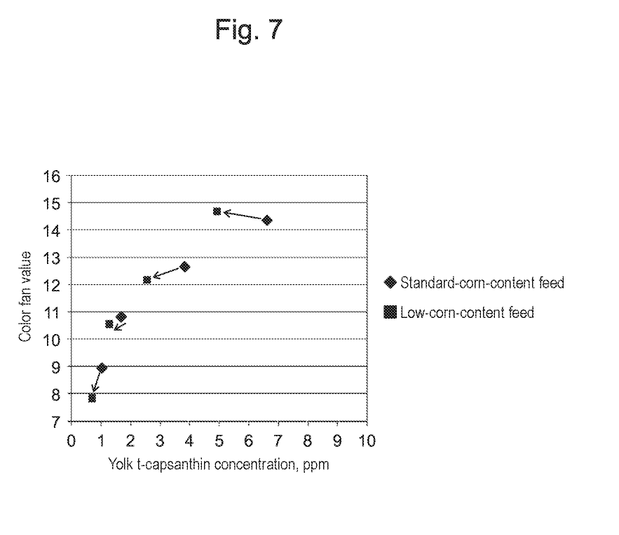

FIGS. 6 and 7 show the relationship between the astaxanthin and t-capsanthin concentrations in the yolk of eggs obtained in Example 1 (the concentrations of astaxanthin and t-capsanthin added to the feed were 1, 2, 4, or 8 ppm) and the yolk color fan value.

The results of FIGS. 6 and 7 confirmed that as the yolk astaxanthin or t-capsanthin concentration increased, the color fan value increased in both cases of the standard-corn-content feed and the low-corn-content feed.

In addition, as indicated by arrows in the figures, even at the same concentration of the colorant added, the color fan value tended to increase for astaxanthin in the low-corn-content feed, while the color fan value tended to decrease for t-capsanthin.

### [Example 5] Chicken Egg Color Enhancement Test 2

The effects of the present invention were confirmed using feeds with corn contents of 30% to 50% in the same manner as in Example 1.

Formula feeds were prepared by adding astaxanthin or t-capsanthin to feeds with the compositions listed in Table 5 so as to result in a final concentration of 1 to 16 ppm. In this Example, the feeds were the same except the corn content and the content of brown rice, which is added depending on a decrease in the corn content, and corn-derived corn gluten meal was not mixed therein.

TABLE 5
Feed composition

| Raw material | 30% Corn feed | 40% Corn feed | 50% Corn feed |
|---|---|---|---|
| Corn | 30 | 40 | 50 |
| Grain sorghum | 6.86 | 6.86 | 6.86 |
| Soybean cake | 25.5 | 25.5 | 25.5 |
| Fish meal (CP 60%) | 0.6 | 0.6 | 0.6 |
| Corn gluten meal | — | — | — |
| Brown rice | 20 | 10 | — |
| Defatted rice bran | 4 | 4 | 4 |
| DL-methionine | 0.14 | 0.14 | 0.14 |
| Animal oil and fat | 2.85 | 2.85 | 2.85 |
| Dicalcium phosphate | 1.22 | 1.22 | 1.22 |
| Calcium Carbonate | 8.20 | 8.20 | 8.20 |
| Vitamin, Mineral | 0.63 | 0.63 | 0.63 |

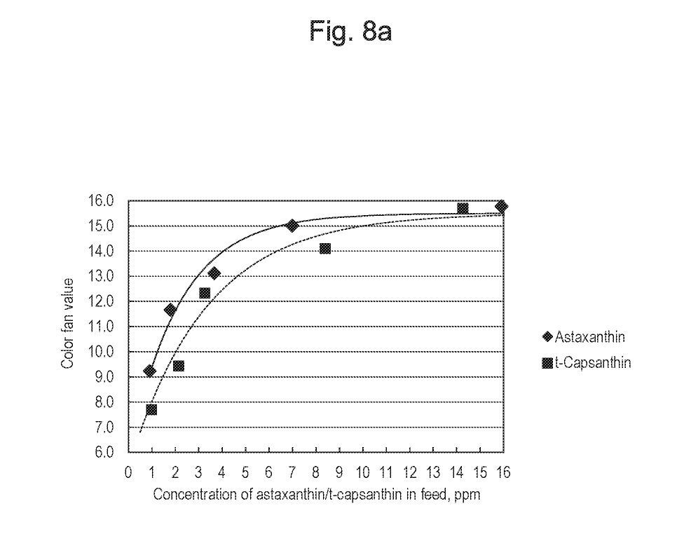

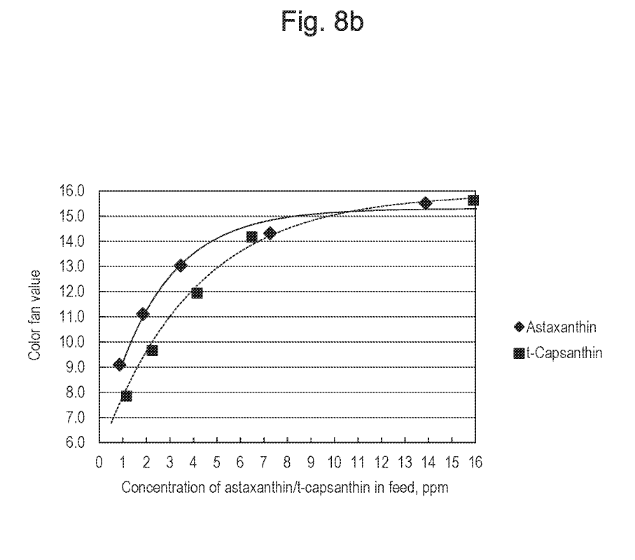

FIGS. 8a and 8b show the relationship between the astaxanthin and t-capsanthin concentrations in the feeds with corn contents of 30% and 50% and the yolk color fan values for the obtained eggs.

As a result, for any of the feeds, the obtained color fan values in the case of using astaxanthin as a coloring agent at a concentration of 1 to 8 ppm were greater than those obtained using t-capsanthin. When each coloring agent was added at a concentration of 16 ppm, no significant difference was observed.

Based on the results of FIGS. 8a and 8b, the concentrations of astaxanthin and t-capsanthin to be added to a feed in order to achieve a desired color fan value of 10 to 15 were calculated for feeds with corn contents of 30% and 50% (Table 6). As shown in Table 6, it was revealed that astaxanthin can provide the comparable color fan value in an amount that is approximately 60% to 80% of the amount of t-capsanthin.

TABLE 6

| | Colorant concentration | | |
|---|---|---|---|
| Desired CF | in feed, ppm | | Astaxanthin/t-Capsanthin |
| value | Astaxanthin | t-Capsanthin | ratio |

30% Corn feed

| | | | |
|---|---|---|---|
| 10 | 1.3 | 2.1 | 62% |
| 11 | 1.7 | 2.8 | 61% |
| 12 | 2.2 | 3.6 | 61% |
| 13 | 3.0 | 4.7 | 64% |
| 14 | 4.0 | 6.4 | 63% |
| 15 | 6.3 | 9.8 | 64% |

50% Corn feed

| | | | |
|---|---|---|---|
| 10 | 1.4 | 2.4 | 58% |
| 11 | 1.9 | 3.1 | 61% |
| 12 | 2.5 | 4.0 | 63% |
| 13 | 3.4 | 5.2 | 65% |
| 14 | 4.7 | 6.8 | 69% |
| 15 | 8.0 | 9.7 | 82% |

The above results revealed that it is possible to reduce the amount of a coloring agent required for color enhancement by choosing astaxanthin as a coloring agent, also for the feeds with corn contents of 30% to 50%. It was also confirmed in this Example that differences in the feed composition resulted in no significant difference in egg production, indicating that egg productivity was maintained.

### [Example 6] Yolk Color Measurement Using Colorimeter 1

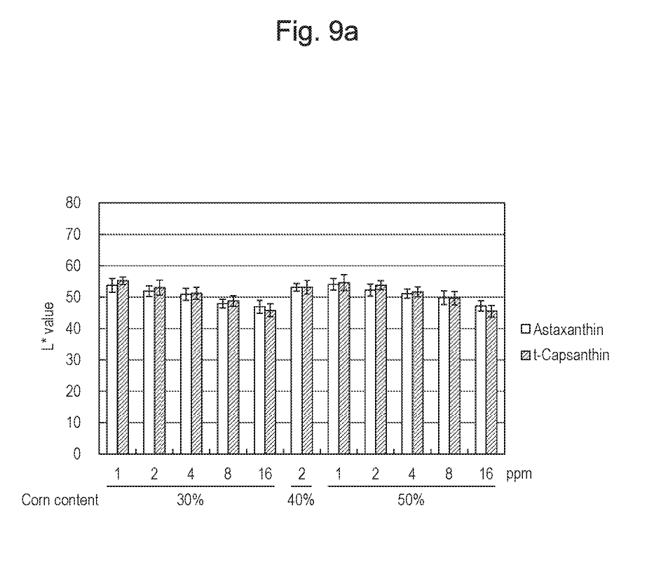

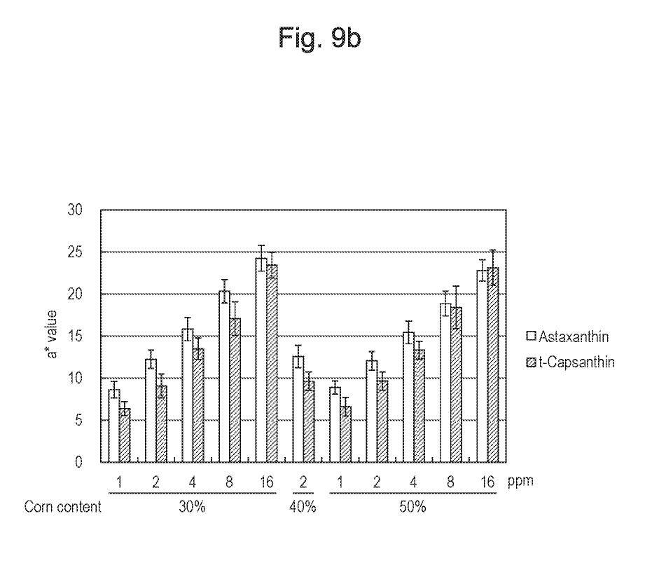

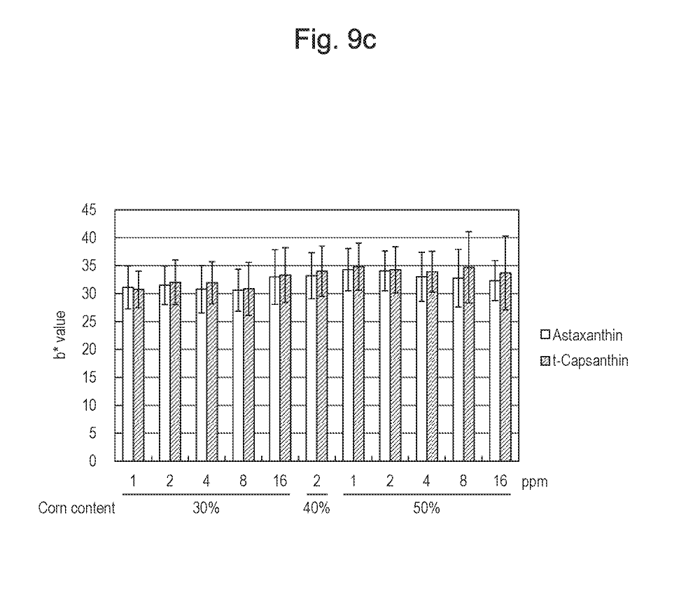

The yolk color was measured using a colorimeter (CM-700d, Konica Minolta Inc.) for eggs obtained in Example 5 and eggs obtained by using formula feeds listed in Table 5, which had been prepared by adding astaxanthin or t-capsanthin to a feed with a corn content of 40% so as to result in a final concentration of 2 ppm. FIGS. 9a to 9c show the measurement results of the L*, a*, and b* values, respectively.

It is known that there is a correlation between the color fan value and the a* value corresponding to red color. The results shown in FIG. 9b indicate that the a* value (red color) increased depending on the amounts of astaxanthin and t-capsanthin added to the feed, and that the a* value obtained with the addition of astaxanthin was greater than that obtained with the addition of t-capsanthin when the amount of the coloring agent was 8 ppm or less. On the other hand, no significant difference was confirmed in terms of the L* value (FIG. 9a) and the b* value (FIG. 9c).

### [Example 7] Comparison of Yolk Carotenoid Content and Composition 2

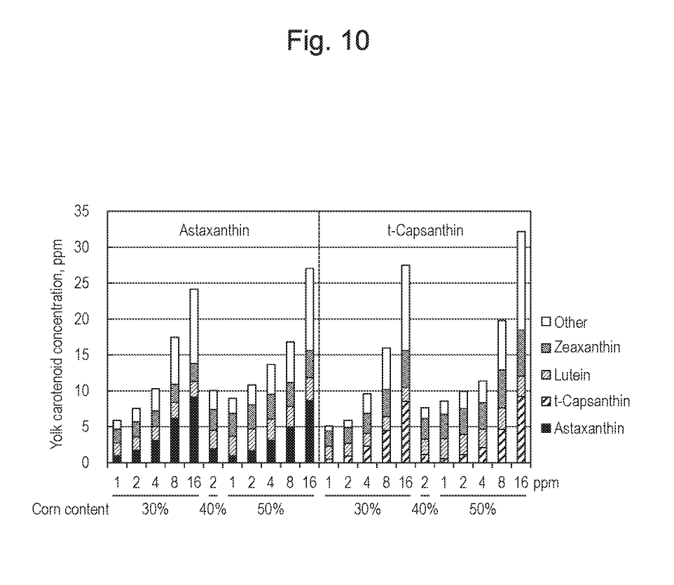

The yolk carotenoid concentration in eggs obtained in Examples 5 and 6 was determined for five eggs for each test plot in the same manner as in Example 1. FIG. 10 shows the yolk carotenoid compositions for eggs collected from chickens that had been fed with the formula feeds with corn contents of 30% to 50% comprising a colorant added at a concentration of 1 to 16 ppm for 2 to 4 weeks.

As is apparent from the figure, the yolk carotenoid concentration increased depending on the concentrations of astaxanthin and t-capsanthin added.

It was also confirmed that the amount of astaxanthin accumulated in the yolk tended to increase as the corn content decreased. Meanwhile, such tendency was not observed for t-capsanthin.

### [Example 8] Chicken Egg Color Enhancement Test 3

The effects of the present invention were confirmed using feeds with corn contents of 0% to 30% in the same manner as in Example 1.

Formula feeds were prepared by adding astaxanthin or t-capsanthin to feeds with the compositions listed in Table 7 so as to result in a final concentration of 2 ppm or 4 ppm. In this Example, the feeds were the same except the corn content and the content of brown rice, which is added depending on a decrease in the corn content, and corn-derived corn gluten meal was not mixed therein.

TABLE 7
Feed composition

| | | | | | 30% |
|---|---|---|---|---|---|
| | 0% Corn | 3% Corn | 10% Corn | 20% Corn | Corn |
| Raw material | feed | feed | feed | feed | feed |
| Corn | 0 | 3 | 10 | 20 | 30 |
| Grain sorghum | 6.86 | | | | |
| Soybean cake | 25.5 | | | | |
| Fish meal (CP | 0.6 | | | | |
| 60%) | | | | | |
| Corn gluten meal | — | | | | |
| Brown rice | 50 | 47 | 40 | 30 | 20 |

| | |
|---|---|
| Defatted rice | 4 |
| bran | |
| DL-methionine | 0.14 |
| Animal oil and | 2.85 |
| fat | |
| Dicalcium | 1.22 |
| phosphate | |
| Calcium | 8.2 |
| Carbonate | |
| Vitamin, Mineral | 0.63 |

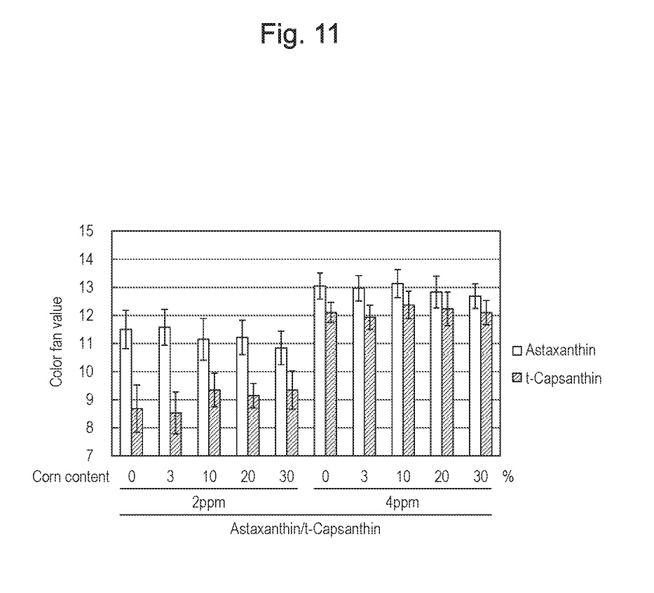

FIG. 11 shows the color fan values of the yolk color for eggs obtained by adding astaxanthin and t-capsanthin to feeds with corn contents of 0% to 30%.

As a result, for each feed, the color fan value achieved by using astaxanthin as a coloring agent was greater than that achieved by using t-capsanthin.

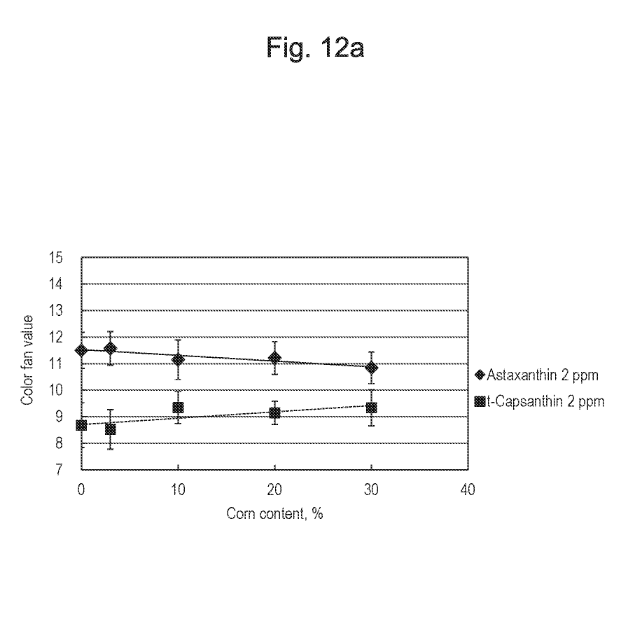

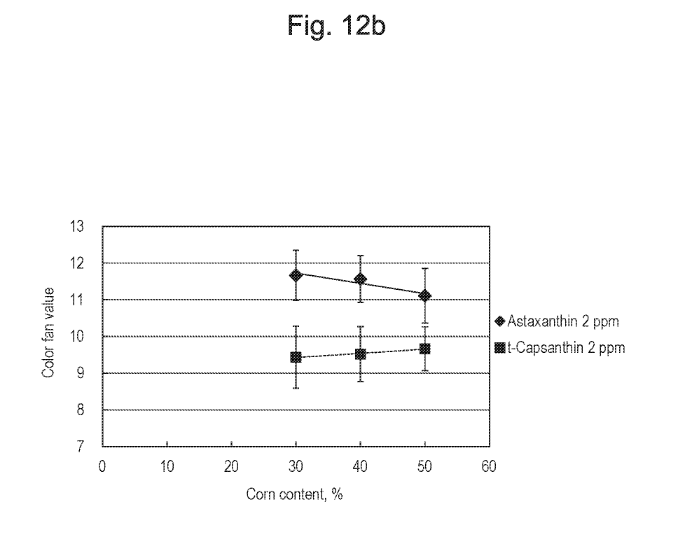

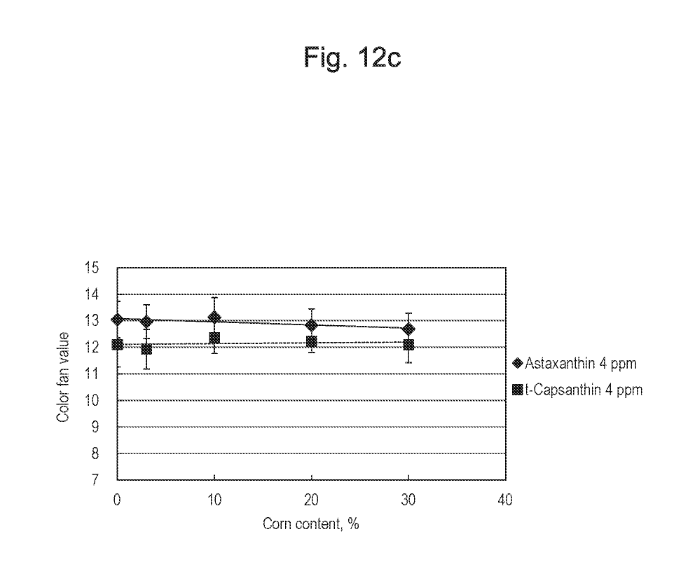

FIGS. 12a to 12c show the yolk color fan values for eggs obtained using the formula feeds prepared by adding a coloring agent to feeds with corn contents of 0% to 30% and feeds with corn contents of 30% to 50% at a concentration of 2 ppm or 4 ppm, based on the above results and the results obtained in Example 5 (FIGS. 8a and 8b). As is apparent from the figures, it was shown that, by using the formula feed to which the coloring agent has been added at a concentration of 2 ppm or 4 ppm, the formula feed of the present invention can provide eggs with color fan values significantly higher than those achieved by adding t-capsanthin.

### [Example 9] Comparison of Yolk Carotenoid Content and Composition 3

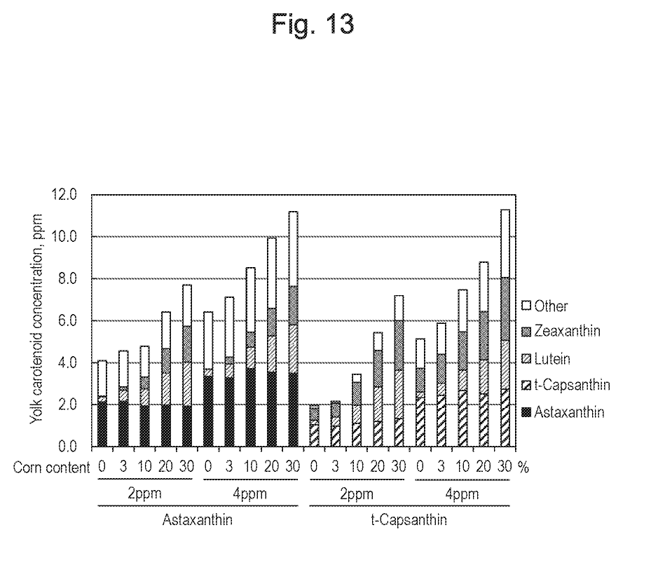

The yolk carotenoid concentration in eggs obtained in Example 8 was determined for five eggs for each test plot in the same manner as in Example 1. FIG. 13 shows the yolk carotenoid compositions for eggs collected from chickens that had been fed with the formula feeds with corn contents of 0% to 30% comprising a colorant added at a concentration of 2 ppm or 4 ppm for 2 to 4 weeks.

As is apparent from the figure, the carotenoid concentration increased depending on the corn content, and the yolk carotenoid concentration also increased depending on the concentrations of astaxanthin and t-capsanthin added. Astaxanthin was transferred to the yolk at a concentration greater than that of t-capsanthin under any conditions.

### [Example 10] Yolk Color Measurement Using Colorimeter 2

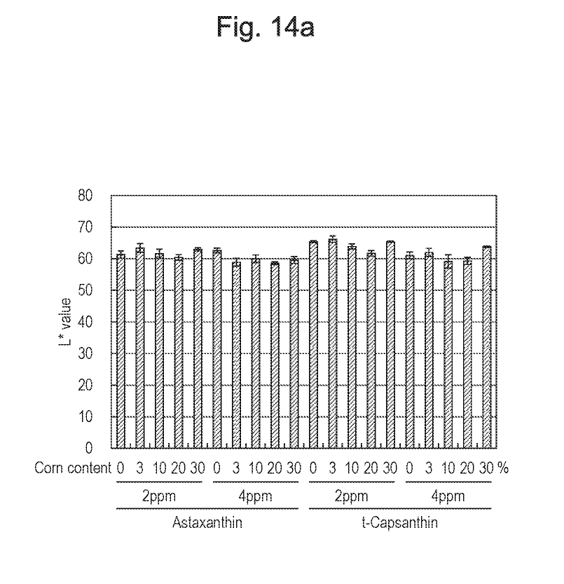

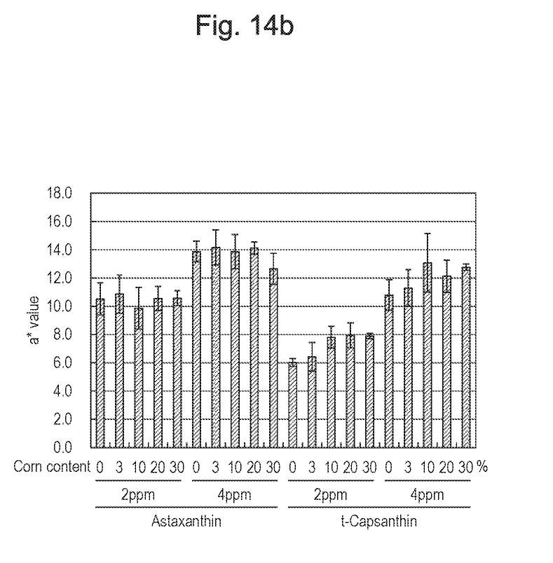

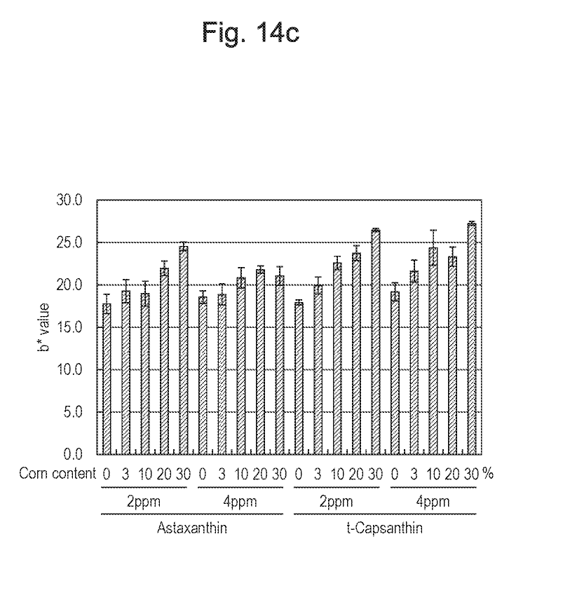

The yolk color of eggs obtained in Example 8 was measured using a colorimeter (CM-700d, Konica Minolta Inc.). FIGS. 14a to 14c show the measurement results of the L*, a*, and b* values, respectively.

The results shown in FIG. 14b indicate that the a* value (red color) increases depending on the amounts of astaxanthin and t-capsanthin (2 ppm or 4 ppm) added to feeds. The a* value increased in proportional to the corn content for t-capsanthin. However, variation in the corn content did not cause the a* value to significantly vary in the case of adding astaxanthin. Meanwhile, there was no significant difference in the L* value (FIG. 14a) corresponding to brightness, and the b* value (FIG. 14c) corresponding to yellow color increased in response to the corn content, which was considered to be a change caused by the corn-derived yellow colorant.

### INDUSTRIAL APPLICABILITY

There has been no obvious fluctuation in the egg price for several decades and the egg price has been changing within a low price range. Therefore, chicken farmers are required to provide eggs with high marketability while maintaining their low prices in the highly competitive market. For such reason, it is very meaningful to reduce the cost of feeds. In addition, it becomes possible to eventually reflect cost reduction on the egg price in the market.

All publications, patents, and patent applications cited herein are incorporated herein by reference in their entirety.

1. A formula feed for poultry, in which the content of corn is 50% or less and the content of astaxanthin from a dry powder of a bacterium is 1 to 8 ppm.

2. The formula feed according to claim 1, wherein the bacterium is a microorganism of the genus Paracoccus.

3. The formula feed according to claim 1, wherein the content of rice, wheat, barley, soybean, milo, and/or raw materials therefrom is 10% or more.

4. A method of obtaining eggs taking on a yolk color corresponding to a color fan value of 9 to 15 by raising poultry using a formula feed, in which the content of corn is 50% or less and the content of astaxanthin from a dry powder of a bacterium is 1 to 8 ppm.

5. The method according to claim 4, which comprises supplying the formula feed for 2 weeks or longer.

6. The method according to claim 4, wherein the bacterium is a microorganism of the genus Paracoccus.

7. A poultry egg taking on a yolk color corresponding to a color fan value of 9 to 15, which is obtained by raising poultry using a formula feed, in which the content of corn is 50% or less and the content of astaxanthin from a dry powder of a bacterium is 1 to 8 ppm.

8. The poultry egg according to claim 7, wherein the bacterium is a microorganism of the genus Paracoccus.

9. The poultry egg according to claim 7, wherein the concentration of astaxanthin in the yolk is 12 ppm or less.

10. The formula feed according to claim 2, wherein the content of rice, wheat, barley, soybean, milo, and/or raw materials therefrom is 10% or more.

11. The method according to claim 5, wherein the bacterium is a microorganism of the genus Paracoccus.

12. The poultry egg according to claim 8, wherein the concentration of astaxanthin in the yolk is 12 ppm or less.
